(19) United States
(12) Patent Application Publication
(10) Pub. No.: US 2019/0160268 A1
(43) Pub. Date: May 30, 2019

(54) GUIDE CATHETER FOR DILATION SYSTEM

(71) Applicant: Acclarent, Inc., Irvine, CA (US)

(72) Inventors: Don Q. Ngo-Chu, Irvine, CA (US); Tuan Pham, Huntington Beach, CA (US); John H. Thinnes, Mission Viejo, CA (US); George L. Matlock, Pleasanton, CA (US); Todd A. Veloni, Lake Forest, CA (US); Ketan P. Muni, San Jose, CA (US); Amit A. More, Irvine, CA (US)

(21) Appl. No.: 15/822,489

(22) Filed: Nov. 27, 2017

(51) Int. Cl. A61M 29/02; A61B 1/07; A61M 25/09; A61B 1/00; A61M 3/02; A61M 25/06

(52) U.S. Cl. CPC A61M 29/02; A61B 1/07; A61M 25/09; A61M 2210/0681; A61M 3/0279; A61M 25/0662; A61M 2205/0266; A61B 1/00195

(57) ABSTRACT

An apparatus includes a dilation catheter and a guide catheter. The dilation catheter includes an expandable dilator that is configured to transition between a non-expanded state and an expanded state. The guide catheter includes a hollow shaft and a malleable portion. The hollow shaft and the malleable portion together define a lumen in which the dilation catheter is slidably disposed. The malleable portion is configured to selectively bend relative to the hollow shaft from a first angular configuration to a second angular configuration. The malleable portion is configured to be coaxial with the longitudinal axis in the first angular configuration. The malleable portion is configured to define an angle that is oblique or perpendicular with the longitudinal axis in the second angular configuration. The malleable portion rigid enough to maintain a selected bend angle relative to the hollow shaft while inserted within a nasal cavity.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guide wire and guide catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Irvine, Calif.

A variable direction view endoscope may be used with such a system to provide visualization within the anatomical passageway (e.g., the ear, nose, throat, paranasal sinuses, etc.) to position the balloon at desired locations. A variable direction view endoscope may enable viewing along a variety of transverse viewing angles without having to flex the shaft of the endoscope within the anatomical passageway. Such an endoscope that may be provided in accordance with the teachings of U.S. Pub. No. 2010/0030031, entitled “Swing Prism Endoscope,” published Feb. 4, 2010, the disclosure of which is incorporated by reference herein.

While a variable direction view endoscope may be used to provide visualization within the anatomical passageway, it may also be desirable to provide additional visual confirmation of the proper positioning of the balloon before inflating the balloon. This may be done using an illuminating guidewire. Such a guidewire may be positioned within the target area and then illuminated, with light projecting from the distal end of the guidewire. This light may illuminate the adjacent tissue (e.g., hypodermis, subdermis, etc.) and thus be visible to the naked eye from outside the patient through transcutaneous illumination. For instance, when the distal end is positioned in the maxillary sinus, the light may be visible through the patient's cheek. Using such external visualization to confirm the position of the guidewire, the balloon may then be advanced distally along the guidewire into position at the dilation site. Such an illuminating guidewire may be provided in accordance with the teachings of U.S. Pub. No. 2012/0078118, entitled “Sinus Illumination Lightwire Device,” published Mar. 29, 2012, the disclosure of which is incorporated by reference herein. An example of such an illuminating guidewire is the Relieva Luma Sentry™ Sinus Illumination System by Acclarent, Inc. of Irvine, Calif.

It may be desirable to provide easily controlled placement of a balloon in dilation procedures, including procedures that will be performed only by a single operator. While several systems and methods have been made and used to inflate an inflatable member such as a dilation balloon, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a side elevational view of an exemplary dilation catheter system;

FIG. 2A depicts a side elevational view of an exemplary illuminating guidewire of the dilation catheter system of FIG. 1;

FIG. 2B depicts a side elevational view of an exemplary guide catheter of the dilation catheter system of FIG. 1;

FIG. 2C depicts a side elevational view of an exemplary dilation catheter of the dilation catheter system of FIG. 1;

FIG. 3 depicts a detailed side elevational view of the illuminating guide wire of FIG. 2A;

FIG. 4 depicts a detailed side cross-sectional view of the illuminating guidewire of FIG. 2A;

FIG. 5 depicts a perspective view of an exemplary endoscope suitable for use with the dilation catheter system of FIG. 1;

FIG. 6 depicts a side elevational view of the distal end of the endoscope of FIG. 5, showing an exemplary range of viewing angles;

FIG. 7A depicts a front view of the guide catheter of FIG. 2B positioned adjacent an ostium of the maxillary sinus;

FIG. 7B depicts a front view of the guide catheter of FIG. 2B positioned adjacent an ostium of the maxillary sinus, with the dilation catheter of FIG. 2C and the illuminating guidewire of FIG. 2A positioned in the guide catheter and a distal portion of the guidewire positioned in the maxillary sinus;

FIG. 7C depicts a front view of the guide catheter of FIG. 2B positioned adjacent an ostium of the maxillary sinus, with the illuminating guidewire of FIG. 2A translated further distally relative to the guide catheter and into the maxillary sinus;

FIG. 7D depicts a front view of the guide catheter of FIG. 2B positioned adjacent an ostium of the maxillary sinus, with the dilation catheter of FIG. 2C translated distally relative to the guide catheter along the illuminating guidewire of FIG. 2A so as to position a balloon of the dilation catheter within the ostium;

FIG. 7E depicts a front view of an ostium of the maxillary sinus, with the ostium having been enlarged by inflation of the balloon of FIG. 7D;

FIG. 8 depicts a side elevational view of an array of alternative reusable guide catheters that may be used with the dilation catheter system of FIG. 1;

FIG. 9 depicts a side elevational view of a distal end of an alternative reusable guide catheter that may be used with the dilation catheter system of FIG. 1;

FIG. 10 depicts a perspective view of the distal end of the reusable guide catheter of FIG. 9;

FIG. 11 depicts a side elevational view of a distal end of another alternative reusable guide catheter than may be used with the dilation catheter system of FIG. 1;

FIG. 12 depicts a perspective view of the distal end of the reusable guide catheter of FIG. 11;

FIG. 13A depicts a side elevational view of another alternative reusable guide catheter that may be used with a dilation catheter system of FIG. 1, where the guide catheter has a malleable distal end in a straight configuration;

FIG. 13B depicts a side elevational view of the reusable guide catheter of FIG. 13A, where the malleable distal end is in a first bent configuration; and

FIG. 13C depicts a side elevational view of the reusable guide catheter of FIG. 13A, where the malleable distal end is in a second bent configuration.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Overview of Exemplary Dilation Catheter System

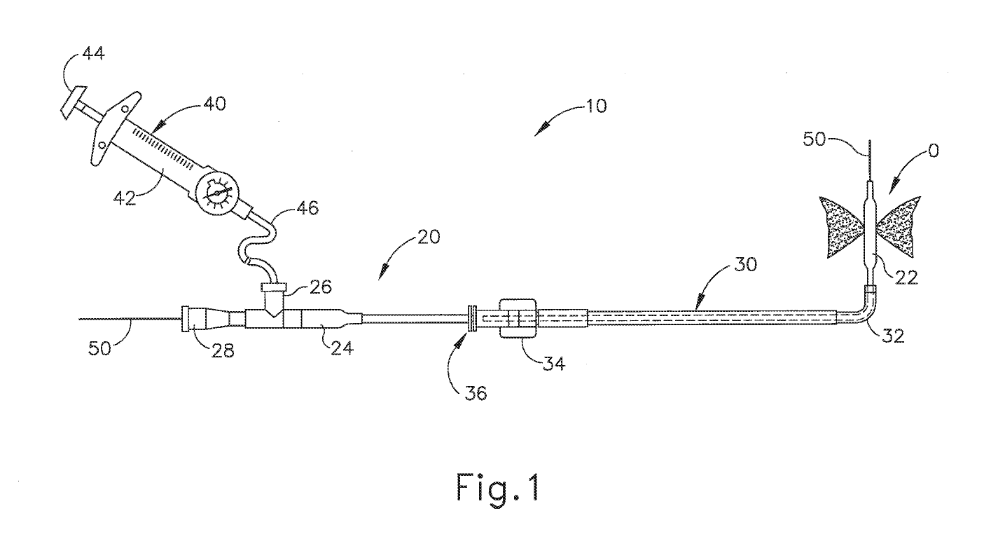

FIG. 1 shows an exemplary dilation catheter system (10) that may be used to dilate the ostium of a paranasal sinus; or to dilate some other anatomical passageway (e.g., within the ear, nose, or throat, etc.). Dilation catheter system (10) of this example comprises a dilation catheter (20), a guide catheter (30), an inflator (40), and a guidewire (50). By way of example only, dilation catheter system (10) may be configured in accordance with at least some of the teachings of U.S. Patent Pub. No. 2011/0004057, the disclosure of which is incorporated by reference herein. In some versions, at least part of dilation catheter system (10) is configured similar to the Relieva® Spin Balloon Sinuplasty™ System by Acclarent, Inc. of Irvine, Calif.

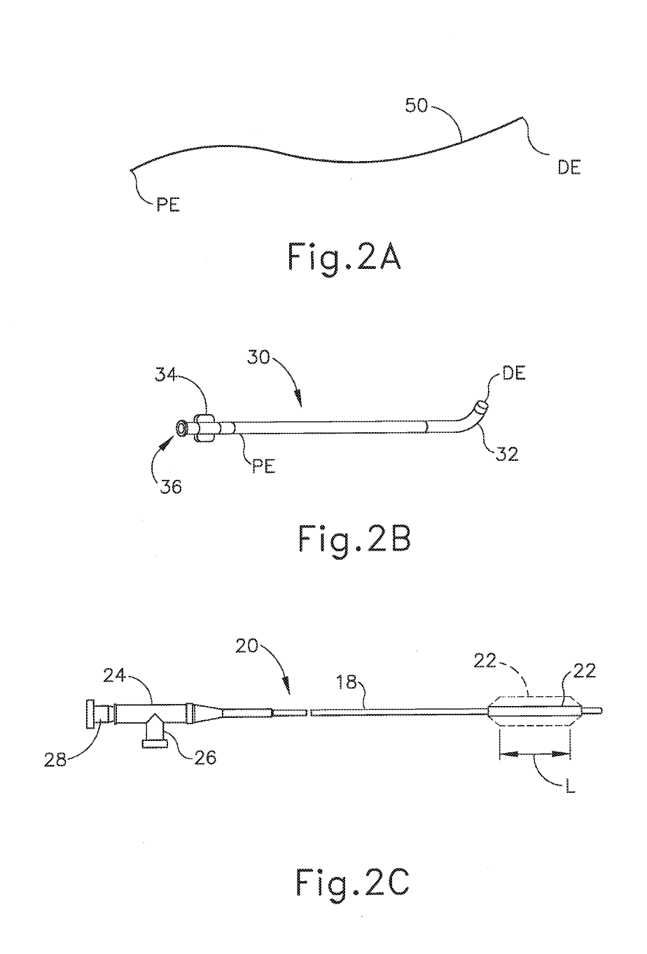

As best seen in FIG. 2C, the distal end (DE) of dilation catheter (20) includes an inflatable dilator (22). The proximal end (PE) of dilation catheter (20) includes a grip (24), which has a lateral port (26) and an open proximal end (28). A hollow-elongate shaft (18) extends distally from grip (24). Dilation catheter (20) includes a first lumen (not shown) formed within shaft (18) that provides fluid communication between lateral port (26) and the interior of dilator (22). Dilator catheter (20) also includes a second lumen (not shown) formed within shaft (18) that extends from open proximal end (28) to an open distal end that is distal to dilator (22). This second lumen is configured to slidably receive guidewire (50). The first and second lumens of dilator catheter (20) are fluidly isolated from each other. Thus, dilator (22) may be selectively inflated and deflated by communicating fluid along the first lumen via lateral port (26) while guidewire (50) is positioned within the second lumen. In some versions, dilator catheter (20) is configured similar to the Relieva Ultirra™ Sinus Balloon Catheter by Acclarent, Inc. of Irvine, Calif. In some other versions, dilator catheter (20) is configured similar to the Relieva Solo Pro™ Sinus Balloon Catheter by Acclarent, Inc. of Irvine, Calif. Other suitable forms that dilator catheter (20) may take will be apparent to those of ordinary skill in the art in view of the teachings herein.

As best seen in FIG. 2B, guide catheter (30) of the present example includes a bent distal portion (32) at its distal end (DE) and a grip (34) at its proximal end (PE). Grip (34) has an open proximal end (36). Guide catheter (30) defines a lumen that is configured to slidably receive dilation catheter (20), such that guide catheter (30) may guide dilator (22) out through bent distal end (32). In some versions, guide catheter (30) is configured similar to the Relieva Flex™ Sinus Guide Catheter by Acclarent, Inc. of Irvine, Calif. Other suitable forms that guide catheter (30) may take will be apparent to those of ordinary skill in the art in view of the teachings herein.

Referring back to FIG. 1, inflator (40) of the present example comprises a barrel (42) that is configured to hold fluid and a plunger (44) that is configured to reciprocate relative to barrel (42) to selectively discharge fluid from (or draw fluid into) barrel (42). Barrel (42) is fluidly coupled with lateral port (26) via a flexible tube (46). Thus, inflator (40) is operable to add fluid to dilator (22) or withdraw fluid from dilator (22) by translating plunger (44) relative to barrel (42). In the present example, the fluid communicated by inflator (40) comprises saline, though it should be understood that any other suitable fluid may be used. There are various ways in which inflator (40) may be filled with fluid (e.g., saline, etc.). By way of example only, before flexible tube (46) is coupled with lateral port (26), the distal end of flexible tube (46) may be placed in a reservoir containing the fluid. Plunger (44) may then be retracted from a distal position to a proximal position to draw the fluid into barrel (42). Inflator (40) may then be held in an upright position, with the distal end of barrel (42) pointing upwardly, and plunger (44) may then be advanced to an intermediate or slightly distal position to purge any air from barrel (42). The distal end of flexible tube (46) may then be coupled with lateral port (26). In some versions, inflator (40) is constructed and operable in accordance with at least some of the teachings of U.S. Pub. No. 2014/0074141, entitled “Inflator for Dilation of Anatomical Passageway,” published Mar. 13, 2014, the disclosure of which is incorporated by reference herein.

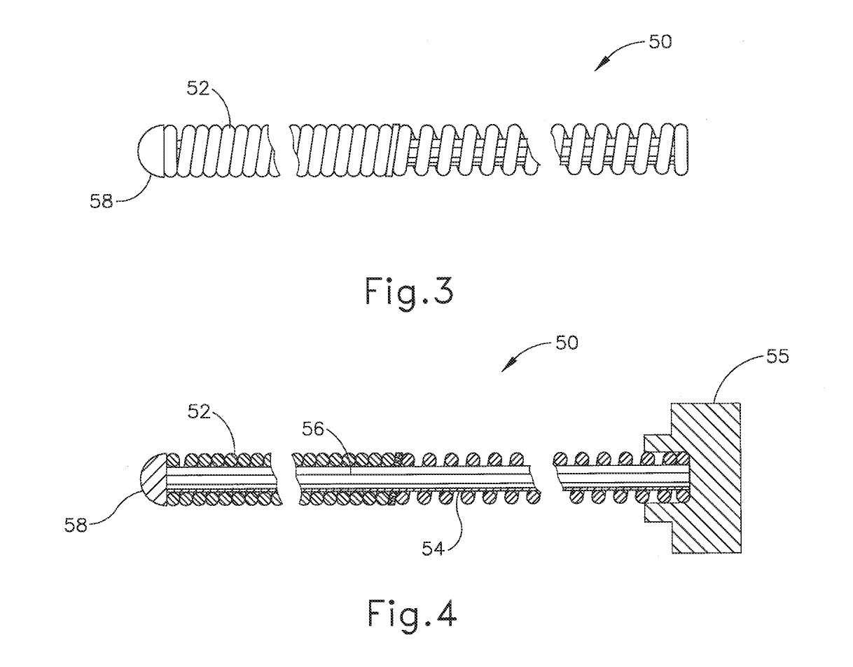

As shown in FIGS. 2A, 3, and 4, guidewire (50) of the present example comprises a coil (52) positioned about a core wire (54). An illumination fiber (56) extends along the interior of core wire (54) and terminates in an atraumatic lens (58). A connector (55) at the proximal end of guidewire (50) enables optical coupling between illumination fiber (56) and a light source (not shown). Illumination fiber (56) may comprise one or more optical fibers. Lens (58) is configured to project light when illumination fiber (56) is illuminated by the light source, such that illumination fiber (56) transmits light from the light source to the lens (58). In some versions, the distal end of guidewire (50) is more flexible than the proximal end of guidewire (50). Guidewire (50) has a length enabling the distal end of guidewire (50) to be positioned distal to dilator (22) while the proximal end of guidewire (50) is positioned proximal to grip (24). Guidewire (50) may include indicia along at least part of its length (e.g., the proximal portion) to provide the operator with visual feedback indicating the depth of insertion of guidewire (50) relative to dilation catheter (20). By way of example only, guidewire (50) may be configured in accordance with at least some of the teachings of U.S. Pub. No. 2012/0078118, the disclosure of which is incorporated by reference herein. In some versions, guidewire (50) is configured similar to the Relieva Luma Sentry™ Sinus Illumination System by Acclarent, Inc. of Irvine, Calif. Other suitable forms that guidewire (50) may take will be apparent to those of ordinary skill in the art in view of the teachings herein.

II. Overview of Exemplary Endoscope

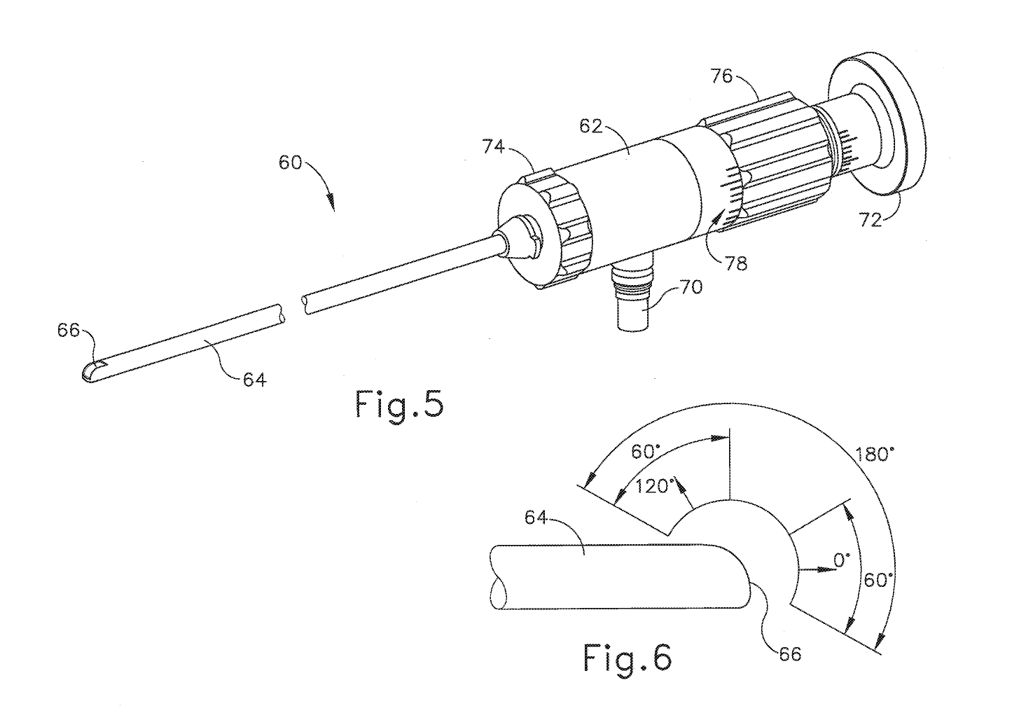

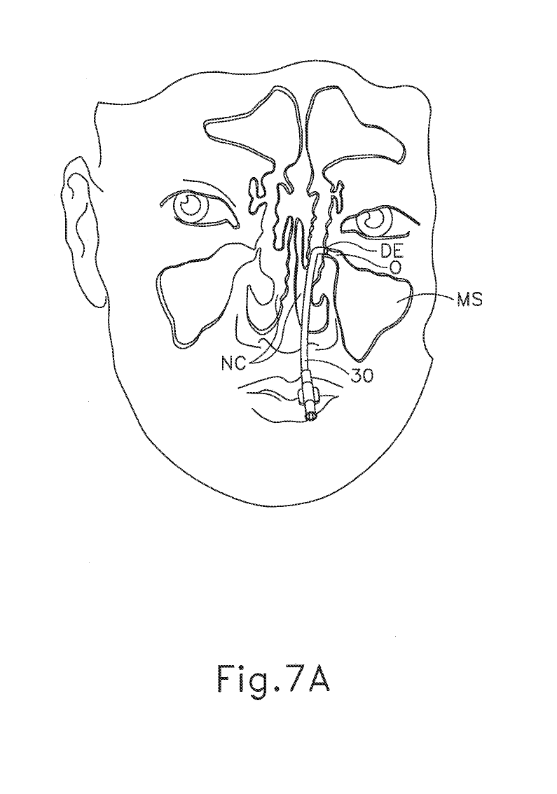

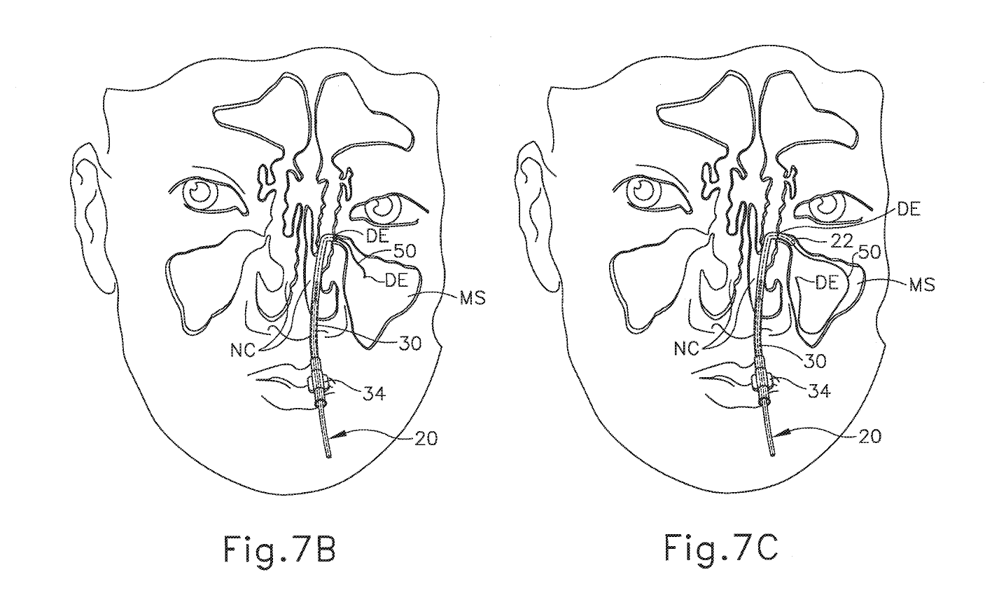

As noted above, an endoscope (60) may be used to provide visualization within an anatomical passageway (e.g., within the nasal cavity, etc.) during a process of using dilation catheter system (10). As shown in FIGS. 4-5, endoscope of the present example comprises a body (62) and a rigid shaft (64) extending distally from body (62). The distal end of shaft (64) includes a curved transparent window (66). A plurality of rod lenses and light transmitting fibers may extend along the length of shaft (64). A lens is positioned at the distal end of the rod lenses and a swing prism is positioned between the lens and window (66). The swing prism is pivotable about an axis that is transverse to the longitudinal axis of shaft (64). The swing prism defines a line of sight that pivots with the swing prism. The line of sight defines a viewing angle relative to the longitudinal axis of shaft (64). This line of sight may pivot from approximately 0 degrees to approximately 120 degrees, from approximately 10 degrees to approximately 90 degrees, or within any other suitable range. The swing prism and window (66) also provide a field of view spanning approximately 60 degrees (with the line of sight centered in the field of view). Thus, the field of view enables a viewing range spanning approximately 180 degrees, approximately 140 degrees, or any other range, based on the pivot range of the swing prism. Of course, all of these values are mere examples.

Body (62) of the present example includes a light post (70), an eyepiece (72), a rotation dial (74), and a pivot dial (76). Light post (70) is in communication with the light transmitting fibers in shaft (64) and is configured to couple with a source of light, to thereby illuminate the site in the patient distal to window (66). Eyepiece (72) is configured to provide visualization of the view captured through window (66) via the optics of endoscope (60). It should be understood that a visualization system (e.g., camera and display screen, etc.) may be coupled with eyepiece (72) to provide visualization of the view captured through window (66) via the optics of endoscope (60). Rotation dial (74) is configured to rotate shaft (64) relative to body (62) about the longitudinal axis of shaft (64). It should be understood that such rotation may be carried out even while the swing prism is pivoted such that the line of sight is non-parallel with the longitudinal axis of shaft (64). Pivot dial (76) is coupled with the swing prism and is thereby operable to pivot the swing prism about the transverse pivot axis. Indicia (78) on body (62) provide visual feedback indicating the viewing angle. Various suitable components and arrangements that may be used to couple rotation dial (74) with the swing prism will be apparent to those of ordinary skill in the art in view of the teachings herein. By way of example only, endoscope (60) may be configured in accordance with at least some of the teachings of U.S. Pub. No. 2010/0030031, the disclosure of which is incorporated by reference herein. Other suitable forms that endoscope (60) may take will be apparent to those of ordinary skill in the art in view of the teachings herein

III. Exemplary Method for Dilating the Ostium of a Maxillary Sinus

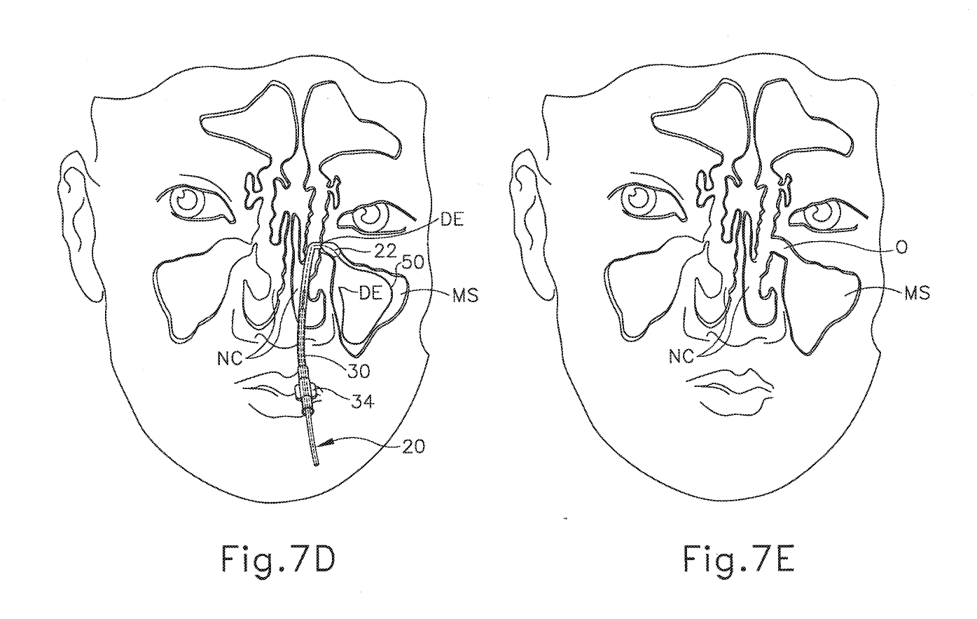

FIGS. 7A-7E show an exemplary method for using dilation catheter system (10) discussed above to dilate a sinus ostium (O) of a maxillary sinus (MS) of a patient. While the present example is being provided in the context of dilating a sinus ostium (O) of a maxillary sinus (MS), it should be understood that dilation catheter system (10) may be used in various other procedures. By way of example only, dilation catheter system (10) and variations thereof may be used to dilate a Eustachian tube, a larynx, a choana, a sphenoid sinus ostium, one or more openings associated with one or more ethmoid sinus air cells, the frontal recess, and/or other passageways associated with paranasal sinuses. Other suitable ways in which dilation catheter system (10) may be used will be apparent to those of ordinary skill in the art in view of the teachings herein.

In the procedure of the present example, guide catheter (30) may be inserted transnasally and advanced through the nasal cavity (NC) to a position within or near the targeted anatomical passageway to be dilated, the sinus ostium (O), as shown in FIG. 7A. Inflatable dilator (22) and the distal end of guidewire (50) may be positioned within or proximal to bent distal end (32) of guide catheter (30) at this stage. This positioning of guide catheter (30) may be verified endoscopically with an endoscope such as endoscope (60) described above and/or by direct visualization, radiography, and/or by any other suitable method. After guide catheter (30) has been positioned, the operator may advance guidewire (50) distally through guide catheter (30) such that a distal portion of the guidewire (50) passes through the ostium (O) of the maxillary sinus (MS) and into the cavity of the maxillary sinus (MS) as shown in FIGS. 7B and 7C. The operator may illuminate illumination fiber (56) and lens (58), which may provide transcutaneous illumination through the patient's face to enable the operator to visually confirm positioning of the distal end of guidewire (50) in the maxillary sinus (MS) with relative ease.

As shown in FIG. 7C, with guide catheter (30) and guidewire (50) suitably positioned, dilation catheter (20) is advanced along guidewire (50) and through bent distal end (32) of guide catheter (30), with dilator (22) in a non-dilated state until dilator (22) is positioned within the ostium (O) of the maxillary sinus (MS) (or some other targeted anatomical passageway). After dilator (22) has been positioned within the ostium (O), dilator (22) may be inflated, thereby dilating the ostium (O), as shown in FIG. 7D. To inflate dilator (22), plunger (44) may be actuated to push saline from barrel (42) of inflator (40) through dilation catheter (20) into dilator (22). The transfer of fluid expands dilator (22) to an expanded state to open or dilate the ostium (O), such as by remodeling the bone, etc., forming ostium (O). By way of example only, dilator (22) may be inflated to a volume sized to achieve about 10 to about 12 atmospheres. Dilator (22) may be held at this volume for a few seconds to sufficiently open the ostium (O) (or other targeted anatomical passageway). Dilator (22) may then be returned to a non-expanded state by reversing plunger (44) of inflator (40) to bring the saline back to inflator (40). Dilator (22) may be repeatedly inflated and deflated in different ostia and/or other targeted anatomical passageways. Thereafter, dilation catheter (20), guidewire (50), and guide catheter (30) may be removed from the patient as shown in FIG. 7E.

In some instances, it may be desirable to irrigate the sinus and paranasal cavity after dilation catheter (20) has been used to dilate the ostium (O). Such irrigation may be performed to flush out blood, etc. that may be present after the dilation procedure. For example, in some cases, guide catheter (30) may be allowed to remain in place after removal of guidewire (50) and dilation catheter (20) and a lavage fluid, other substance, or one or more other devices (e.g., lavage catheters, balloon catheters, cutting balloons, cutters, chompers, rotating cutters, rotating drills, rotating blades, sequential dilators, tapered dilators, punches, dissectors, burs, non-inflating mechanically expandable members, high frequency mechanical vibrators, dilating stents and radiofrequency ablation devices, microwave ablation devices, laser devices, snares, biopsy tools, scopes, and devices that deliver diagnostic or therapeutic agents) may be passed through guide catheter (30) for further treatment of the condition. By way of example only, irrigation may be carried out in accordance with at least some of the teachings of U.S. Pat. No. 7,630,676, entitled “Methods, Devices and Systems for Treatment and/or Diagnosis of Disorders of the Ear, Nose and Throat,” issued Dec. 8, 2009, the disclosure of which is incorporated by reference herein. An example of an irrigation catheter that may be fed through guide catheter (30) to reach the irrigation site after removal of dilation catheter (20) is the Relieva Vortex® Sinus Irrigation Catheter by Acclarent, Inc. of Irvine, Calif. Another example of an irrigation catheter that may be fed through guide catheter (30) to reach the irrigation site after removal of dilation catheter (20) is the Relieva Ultirra® Sinus Irrigation Catheter by Acclarent, Inc. of Irvine, Calif. Of course, irrigation may be provided in the absence of a dilation procedure; and a dilation procedure may be completed without also including irrigation.

IV. Exemplary Alternative Guide Catheters

A. Reusable Guide Catheters

In some instances, it may be desirable to provide a version of guide catheter (30) that is configured to be reused in numerous procedures. For example, it may be desirable to use a guide catheter within a first dilation catheter system (10) during a first procedure, suitably sterilize the same guide catheter using common sterilization methods, and then use the same guide catheter with a second dilation catheter system (10) during a second procedure. Providing a version of guide catheter (30) that may be used for multiple procedures may reduce costs associated with use a dilation catheter system (10) by reducing the cost per procedure.

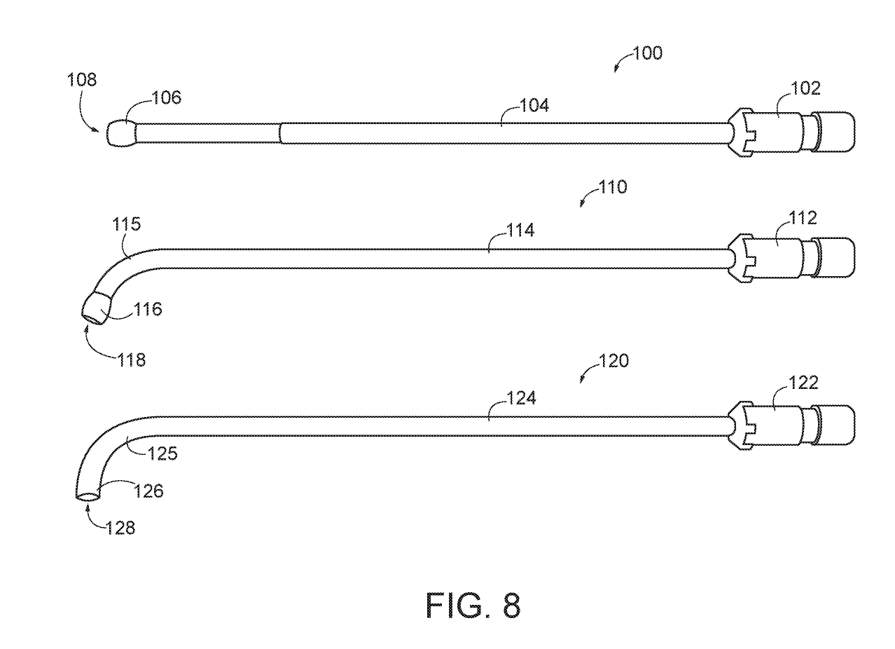

FIG. 8 shows various alternative guide catheters (100, 110, 120) that may each be readily incorporated into dilation catheter system (10) as described above, in place of guide catheter (30). As will be described below, each guide catheter (100, 110, 120) may be used in a first procedure, sterilized using any suitable known sterilization method, and then used for a second procedure. Guide catheters (100, 110, 120) may be used for any suitable number of procedures as would be apparent to one having ordinary skill in the art in view of the teachings herein.

In the present examples, each guide catheter (100, 110, 120) includes a proximal connecting hub (102, 112, 122), respectively. Proximal connecting hubs (102, 112, 122) may allow guide catheter (100, 110, 120) to selectively couple with a suitable handle assembly. By way of example only, guide catheters (100, 110, 120) may selectively couple with a handle assembly (and/or otherwise be configured) in accordance with at least some of the teachings of U.S. Pub. No. 2017/0056632, entitled “Dilation Catheter with Expandable Stop Element,” published Mar. 2, 2017, the disclosure of which is incorporated by reference herein; U.S. Pub. No. 2006/0004323, entitled “Apparatus and Methods for Dilating and Modifying Ostia of Paranasal Sinuses and Other Intranasal or Paranasal Structures,” published Jan. 5, 2006, the disclosure of which is incorporated by reference herein; U.S. Pat. No. 8,894,614, entitled “Devices, Systems, and Methods Useable for Treating Frontal Sinusitis,” issued Nov. 25, 2014, the disclosure of which is incorporated by reference herein; U.S. Pat. No. 7,654,997, entitled “Devices, Systems and Methods for Diagnosing and Treating Sinusitis and Other Disorders of the Ears, Nose and/or Throat,” issued Feb. 2, 2010, the disclosure of which is incorporated by reference herein; and/or U.S. Pat. No. 7,803,150, entitled “Devices, Systems and Methods Useable for Treating Sinusitis,” issued Sep. 28, 2010, the disclosure of which is incorporated by reference herein. In versions where guide catheter (100, 110, 120) is not configured to couple with a handle assembly, guide catheter (100, 110, 120) may include a grip, similar to grip (34) described above.

Additionally, each guide catheter (100, 110, 120) also includes a distally extending hollow shaft (104, 114, 124) that terminates into an open distal tip (106, 116, 126), respectively. Guide catheters (100, 110, 120) each define a lumen (108, 118, 128), respectively. Lumens (108, 118, 128) extend from the proximal end of proximal connecting hubs (102, 112, 122) all the way to open distal tips (106, 116, 126). Therefore, an operator may insert dilation catheter (20) through lumen (108, 118, 128) during exemplary use such that inflatable dilator (22) may traverse through lumen (108, 118, 128) and extend distally past open distal tip (106, 116, 126).

Guide catheters (100, 110) each have open distal tip (106, 116) formed in an atraumatic olive shape. The olive shaped tip, or otherwise bulbous shape, of open distal tip (106, 116) may provide for atraumatic insertion of guide catheters (100, 110) within a patient, respectively. It should be understood that open distal tip (126) may also include an olive shaped tip or otherwise bulbous shaped tip as well.

Guide catheters (110, 120) include bent distal portion (115, 125), respectively. Bent distal portions (115, 125) are bent at an angle relative to a more proximal portion of hollow shafts (114, 124) such that open distal tips (106, 116) may be placed adjacent to a targeted passageway when guide catheter (110, 120) is suitable inserted into a patient. Bent distal portions (115, 125) may form any suitable angle as would be apparent to one having ordinary skill in the art in view of the teachings herein. For instance, each guide catheter (110, 120) may form a bend angle at bent portion (115, 125) that is configured to facilitate access to a particular anatomical structure (e.g., maxillary sinus ostium, sphenoid sinus ostium, frontal recess, Eustachian tube, etc.). Examples of suitable bend angles are described in one or more references cited herein. Other suitable bend angles will be apparent to those of ordinary skill in the art in view of the teachings herein.

In the present example, each guide catheter (100, 110, 120) is rigid along its entire length, such that guide catheters (100, 110, 120) are not malleable or otherwise flexible. In some other versions, at least a distal portion of each guide catheter (100, 110, 120) is malleable. Each guide catheter (100, 110, 120) of the present example is formed from a suitable metal that may allow guide catheter (100, 110, 120) to be sterilized using common sterilization methods. For instance, guide catheters (100, 110, 120) may be made from 316 stainless steel. Therefore, guide catheters (100, 110, 120) may be used in a first procedure, sterilized, and used in a second procedure. Using guide catheter (100, 110, 120) for more than one procedure may reduce the cost per procedure. The inner diameter of each guide catheter (100, 110, 120) may be coated with a lubricious coating to lessen the insertion and retraction force that the inner diameter of guide catheter (100, 110, 120) imparts on dilator (22) when dilation catheter (20) is interested through lumen (108, 118, 128). By way of example only, this lubricious coating may include Poly-Ond® coating by Poly-Plating, Inc. of Chicopee, Mass. Other suitable coatings will be apparent to those of ordinary skill in the art in view of the teachings herein. Alternatively, such a coating may be omitted.

B. Guide Catheters Having Bent Portions with Expanded Inner Diameters

In some instances, the inner diameter of the bent distal portion (32) of guide catheter (30) may undesirably snag, catch, damage or otherwise disrupt dilator (22) or portions of hollow elongate shaft (18) when dilation catheter (20) traverses distally through bent distal portion (32), especially when bent distal portion (32) forms a bend angle that is less than or equal to 90 degrees with a proximal portion of guide catheter (30). Therefore, it may also be desirable to provide a variation of guide catheter (30) that has a larger inner diameter around a bent distal portion. The larger inner diameter may help prevent bent distal portion (32) of guide catheter (30) from undesirably damaging or disrupting dilator (22) or portions of hollow elongate shaft (18) when dilation catheter (20) traverses longitudinally within bent distal portion (32).

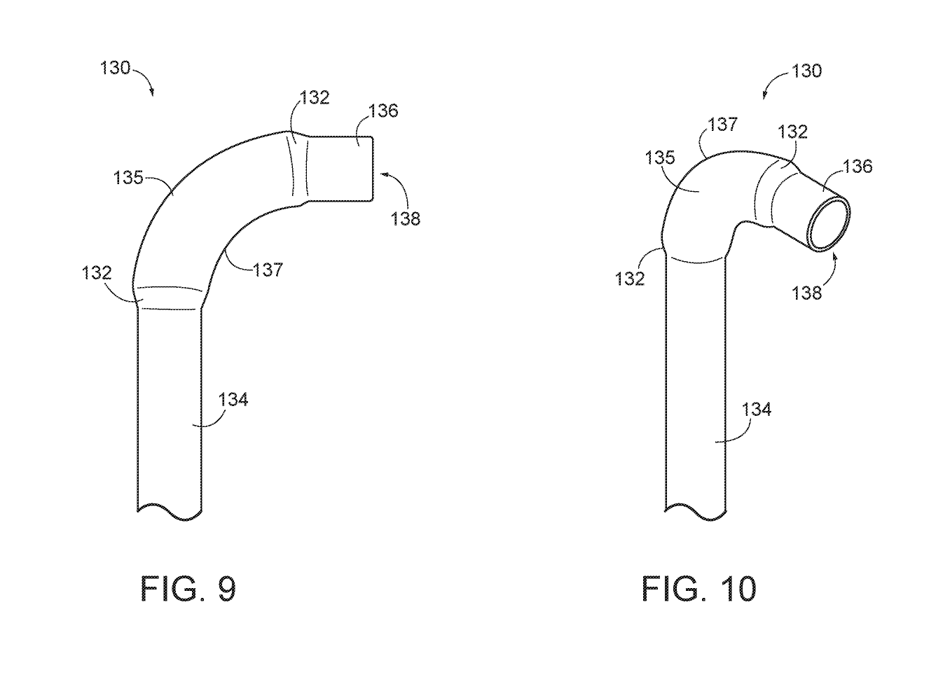

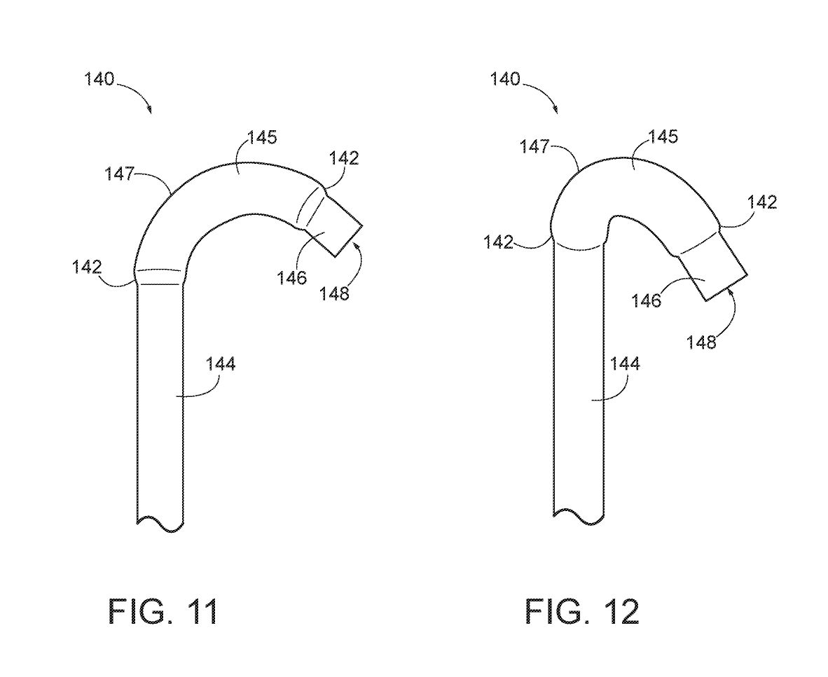

FIGS. 9-10 show an alternative reusable guide catheter (130) that may be readily incorporated into dilation catheter system (10) described above, in place of guide catheter (30). Guide catheter (130) of this example includes a hollow shaft (134) extending into a bent distal portion (135), which then extends into an open distal tip (136). Hollow shaft (134) may proximally connect to a connecting hub or a grip similar to connecting hub (102, 112, 122) or grip (34) described above.

Bent distal portion (135) includes an expanded diameter (137). Bent distal portion (135) is integrally connected to both open distal tip (136) and hollow shaft (134) via tapered transition portions (132). The portion of lumen (138) defined by expanded diameter (137) has a larger inner diameter than the inner diameters of defined by open distal tip (136) and hollow shaft (134). Therefore, when an operator actuates dilator (22) longitudinally through bent distal portion (135), the larger inner diameter provided by expanded diameter (137) may allow for a smooth insertion and retraction of dilator (22) and adjacent portions of hollow elongate shaft (18) through bent distal portion (135). In other words, the larger inner diameter provided by expanded diameter (137) may reduce the chances of guide catheter (130) from undesirably snagging, catching, damaging, or otherwise interfering with dilator (22) and relative portions of hollow elongate shaft (18).

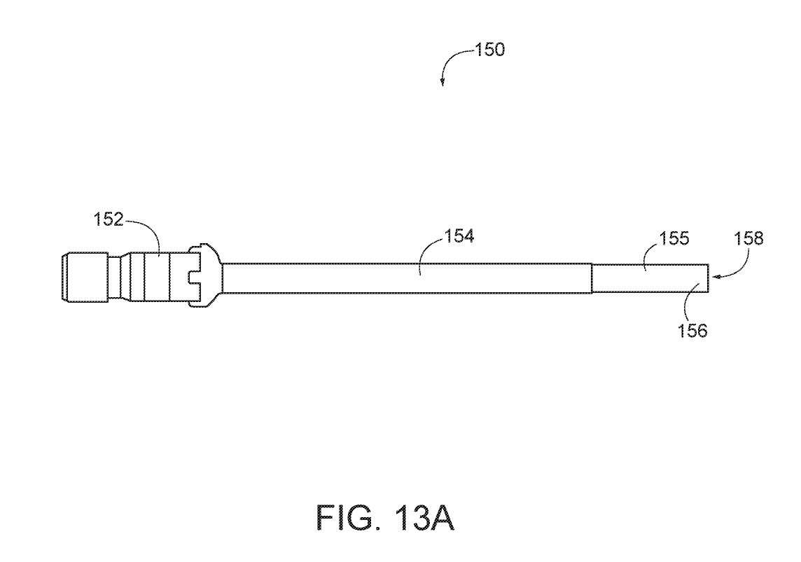

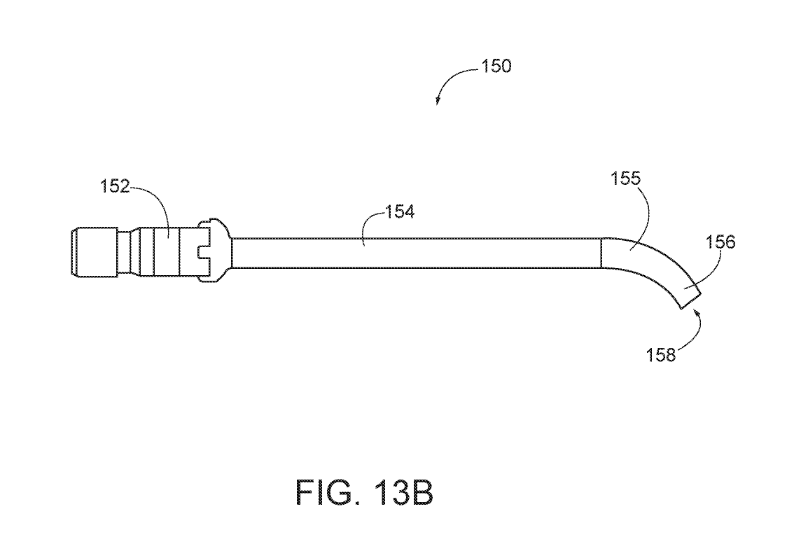

FIGS. 11-12 show an alternative reusable guide catheter (140) that may be readily incorporated into dilation catheter system (10) described above, in place of guide catheter (30). Guide catheter (140) of this example includes a hollow shaft (144) extending into a bent distal portion (145), which then extends into an open distal tip (146). Hollow shaft (144) may proximally connect to a connecting hub or a grip similar to connecting hub (102, 112, 122) or grip (24) described above.

Bent distal portion (145) includes an expanded diameter (147). Bent distal portion (145) is integrally connected to both open distal tip (146) and hollow shaft (144) via tapered transition portions (142). The portion of lumen (148) defined by expanded diameter (147) has a larger inner diameter than the inner diameters of defined by open distal tip (146) and hollow shaft (144). Therefore, when an operator actuates dilator (22) longitudinally through bent distal portion (145), the larger inner diameter provided by expanded diameter (147) may allow for a smooth insertion and retraction of dilator (22) and adjacent portions of hollow elongate shaft (18) through bent distal portion (145). In other words, the larger inner diameter provided by expanded diameter (147) may reduce the chances of guide catheter (140) from undesirably snagging, catching, damaging, or otherwise interfering with dilator (22) and relative portions of hollow elongate shaft (18).

Guide catheter (130, 140) may be formed through a swaging process where all portions of guide catheter (130, 140) start with an initial diameter equal to expanded diameter (137, 147), and the swaging process reduces the diameter of hollow shaft (134, 144) and open distal tip (136, 146), leaving bent portions (135, 145) at the relatively larger diameter (137, 147). Of course, any other suitable manufacturing technique may be utilized as would be apparent to one having ordinary skill in the art in view of the teaching herein.

C. Guide Catheters Having Malleable Distal Portions

In the alternative or in addition to a reusable guide catheter, it may also be desirable to provide a version of guide catheter (30) that is configured to be used in multiple types of procedures. For example, it may be desirable to have a single guide catheter having a malleable distal end that may be selectively bent to various angles prior to being used. Therefore, if an operator desires to access a particular anatomical passageway within a patient, the operator may bend the malleable distal end of the guide catheter to a suitable bend angle for accessing that particular anatomical passageway prior to use of the guide catheter.

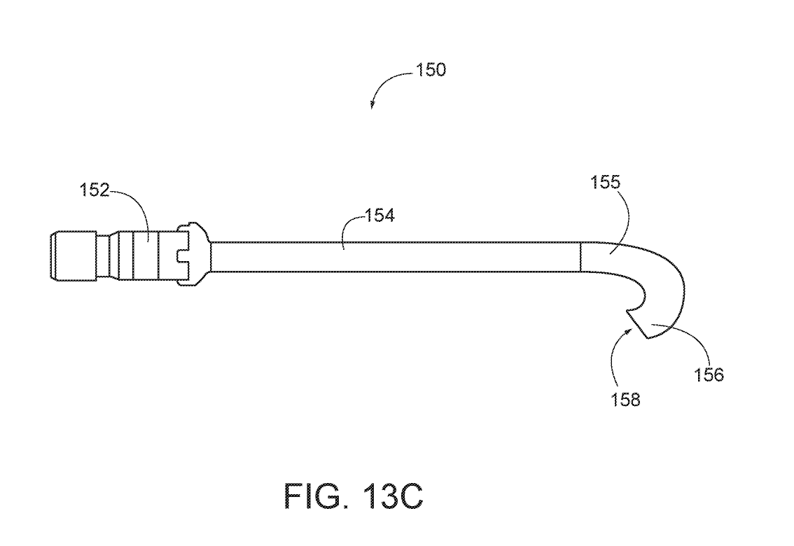

FIGS. 13A-13C show an exemplary alternative guide catheter (150) that may be readily incorporated into dilation catheter system (10) as described above, in place of guide catheter (30). In the present example, guide catheter (150) includes a proximal connecting hub (152). Proximal connecting hub (152) may allow guide catheter (150) to selectively couple with a suitable handle assembly. Alternatively, guide catheter (150) may include a grip, similar to grip (34) described above. Additionally, guide catheter (150) also includes a distally extending hollow shaft (154) that terminates into a malleable distal portion (155) having an open distal tip (156). Guide catheter (150) defines a lumen (158). Lumen (158) extends from the proximal end of proximal connecting hub (152) all the way to open distal tip (156). Therefore, an operator may insert dilation catheter (20) through lumen (158) during exemplary use such that inflatable dilator (22) may traverse through lumen (158) and extend distally past open distal tip (156).

As best seen between FIGS. 13A-13C, malleable distal portion (155) may be selectively bent by an operator to a desired access angle relative to distally extending hollow shaft (154), such that guide catheter (150) may be used to access multiple sinus drainage passageways and/or other anatomical passageways. FIG. 13A shows guide catheter (150) where malleable distal portion (155) is in a straight configuration. FIG. 13B shows guide catheter (150) where malleable distal portion (155) has been bent to form an access angle of around 70 degrees relative to a longitudinal axis of shaft (154). FIG. 13C shows guide catheter (150) where malleable distal portion (1550 has been bent to form an access angle of around 110 degrees relative to a longitudinal axis of shaft (154). It should be understood than any suitable access angle may be formed as would be apparent to one having ordinary skill in the art in view of the teachings herein.

Malleable distal portion (155) is sufficiently rigid such that malleable distal portion (155) may be suitably inserted into a patient near a desired anatomical passageway, with distal portion (155) in a bent or unbent state, without deviating from the chosen bend angle. In particular, once inserted into a patient, malleable distal portion (155) remains fixed in the chosen bend angle. In other words, once inserted into a patient, an operator may not bend malleable distal portion (155) relative to shaft (154); and guide catheter (150) will maintain the selected bend angle at distal portion (155) as dilation catheter (20) traverses guide catheter (150). However, malleable distal portion (150) is sufficiently malleable such that an operator may utilize a bending tool or the strength of their own hand in order to manipulate malleable distal portion (155) to form various access angles as shown in FIGS. 13A-13B. Therefore, malleable distal portion (155) may be bent relative to shaft (154) such that guide catheter (150) may be utilized to access a frontal recess, a sphenoid sinus ostium, a maxillary sinus ostium, a Eustachian tube, etc. Of course, malleable distal portion (155) may be utilized to access any other anatomical passageway as would be apparent to one having ordinary skill in the art in view of the teachings herein.

Malleable distal portion (155) may be formed of shape memory nitinol, stainless steel that is annealed for malleability, a flexible polymer supported by shape memory nitinol wires, or any other suitable material that would be apparent to one having ordinary skill in the art in view of the teachings herein. Additionally, open distal tip (156) may also include an olive shaped tip or other bulbous shaped tip (e.g., as described herein) for atraumatic insertion.

V. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a dilation catheter, wherein the dilation catheter comprises an expandable dilator, wherein the expandable dilator is configured to transition between a non-expanded state and an expanded state, wherein the expandable dilator is configured to fit in an anatomical passageway associated with drainage of a paranasal sinus when the expandable dilator is in the non-expanded state, wherein the expandable dilator is configured to dilate an anatomical passageway associated with drainage of a paranasal sinus when the expandable dilator is in the expanded state; and (b) a guide catheter, wherein the guide catheter comprises: (i) a proximal body, (ii) a hollow shaft extending distally from the proximal body, wherein the hollow shaft defines a longitudinal axis, and (iii) a malleable portion extending distally from the hollow shaft, wherein the proximal body, the hollow shaft, and the malleable portion together define a lumen, wherein the dilation catheter is slidably disposed in the lumen, wherein the malleable portion is dimensioned to be inserted into a nasal cavity, wherein the malleable portion is configured to selectively bend relative to the hollow shaft from a first angular configuration to a second angular configuration, wherein the malleable portion is configured to be coaxial with the longitudinal axis in the first angular configuration, wherein the malleable portion is configured to define an angle that is oblique or perpendicular with the longitudinal axis in the second angular configuration, wherein the malleable portion is sufficiently rigid such that the malleable portion is configured to maintain a selected bend angle relative to the hollow shaft while inserted within the nasal cavity.

### Example 2

The apparatus of Example 1, wherein the malleable portion includes an olive shaped distal tip.

### Example 3

The apparatus of any one or more of Examples 1 through 2, wherein the proximal body includes a connecting hub.

### Example 4

The apparatus of any one or more of Examples 1 through 2, wherein the proximal body includes a grip.

### Example 5

The apparatus of any one or more of Examples 1 through 4, wherein the hollow shaft comprises a 316-stainless steel.

### Example 6

The apparatus of any one or more of Examples 1 through 5, wherein the malleable portion comprises a shape memory nitinol.

### Example 7

The apparatus of any one or more of Examples 1 through 6, wherein the malleable portion comprises an annealed stainless steel.

### Example 8

The apparatus of any one or more of Examples 1 through 7, wherein the malleable portion comprises a flexible polymer supported by a shape memory nitinol wire.

### Example 9

The apparatus of any one or more of Examples 1 through 8, further comprising a shaping tool configured to bend the malleable portion from the first angular configuration to the second angular configuration.

### Example 10

The apparatus of any one or more of Examples 1 through 9, wherein the proximal body and the hollow shaft are made from steel.

### Example 11

The apparatus of any one or more of Examples 1 through 10, wherein a portion of the hollow shaft is dimensioned to be inserted into the nasal cavity.

### Example 12

The apparatus of any one or more of Examples 1 through 11, wherein the shaft defines a first inner diameter, wherein the malleable portion defines a second inner diameter, wherein the second inner diameter is larger than the first inner diameter.

### Example 13

The apparatus of any one or more of Examples 1 through 12, wherein the second angular configuration is around 110 degrees.

### Example 14

The apparatus of any one or more of Examples 1 through 12, wherein the second angular configuration is around 70 degrees.

### Example 15

The apparatus of any one or more of Examples 1 through 14, wherein the hollow shaft also comprises a malleable material.

### Example 16

An apparatus, the apparatus comprising: (a) a dilation catheter, wherein the dilation catheter comprises an expandable dilator, wherein the expandable dilator is configured to transition between a non-expanded state and an expanded state, wherein the expandable dilator is configured to fit in an anatomical passageway associated with drainage of a paranasal sinus when the expandable dilator is in the non-expanded state, wherein the expandable dilator is configured to dilate an anatomical passageway associated with drainage of a paranasal sinus when the expandable dilator is in the expanded state; and (b) a guide catheter, wherein the guide catheter comprises: (i) a hollow shaft, wherein the hollow shaft comprises a first inner diameter having a first dimension, (ii) an open distal tip, and (iii) a bent distal portion extending between the hollow shaft and the open distal tip, wherein the bent distal portion comprises a second inner diameter having a second dimension, wherein the first dimension of the first inner diameter is smaller than the second dimension of the second inner diameter, wherein the hollow shaft, the open distal tip, and the bent distal portion together define a lumen, wherein the lumen includes the first and second inner diameters, wherein the dilation catheter is slidably disposed in the lumen.

### Example 17

The apparatus of Example 16, further comprising a tapered transitioning section coupling the bent distal portion with the hollow shaft.

### Example 18

The apparatus of any one or more of Examples 16 through 17, further comprising a tapered transitioning section coupling the open distal tip with the bent distal portion.

### Example 19

The apparatus of any one or more of Examples 16 through 18, wherein the bent distal portion has an inner diameter of around 0.110 inches.

### Example 20

An apparatus, the apparatus comprising: (a) a dilation catheter, wherein the dilation catheter comprises an expandable dilator, wherein the expandable dilator is configured to transition between a non-expanded state and an expanded state, wherein the expandable dilator is configured to fit in an anatomical passageway associated with drainage of a paranasal sinus when the expandable dilator is in the non-expanded state, wherein the expandable dilator is configured to dilate an anatomical passageway associated with drainage of a paranasal sinus when the expandable dilator is in the expanded state; and (b) a guide catheter, wherein the guide catheter comprises: (i) a proximal body, (ii) a hollow shaft extending distally from the proximal body, and (iii) an open distal tip, wherein the open distal tip has a bulbous configuration, wherein the proximal body, hollow shaft, and distal tip together define a lumen, wherein the dilation catheter is slidably disposed in the lumen, wherein the proximal body, the hollow shaft, and the open distal tip are made from a metallic material.

VI. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a dilation catheter, wherein the dilation catheter comprises an expandable dilator, wherein the expandable dilator is configured to transition between a non-expanded state and an expanded state, wherein the expandable dilator is configured to fit in an anatomical passageway associated with drainage of a paranasal sinus when the expandable dilator is in the non-expanded state, wherein the expandable dilator is configured to dilate an anatomical passageway associated with drainage of a paranasal sinus when the expandable dilator is in the expanded state; and
(b) a guide catheter, wherein the guide catheter comprises:
(i) a proximal body,
(ii) a hollow shaft extending distally from the proximal body, wherein the hollow shaft defines a longitudinal axis, and
(iii) a malleable portion extending distally from the hollow shaft,
wherein the proximal body, the hollow shaft, and the malleable portion together define a lumen, wherein the dilation catheter is slidably disposed in the lumen,
wherein the malleable portion is dimensioned to be inserted into a nasal cavity,
wherein the malleable portion is configured to selectively bend relative to the hollow shaft from a first angular configuration to a second angular configuration, wherein the malleable portion is configured to be coaxial with the longitudinal axis in the first angular configuration, wherein the malleable portion is configured to define an angle that is oblique or perpendicular with the longitudinal axis in the second angular configuration,
wherein the malleable portion is sufficiently rigid such that the malleable portion is configured to maintain a selected bend angle relative to the hollow shaft while inserted within the nasal cavity.

2. The apparatus of claim 1, wherein the malleable portion includes an olive shaped distal tip.

3. The apparatus of claim 1, wherein the proximal body includes a connecting hub.

4. The apparatus of claim 1, wherein the proximal body includes a grip.

5. The apparatus of claim 1, wherein the hollow shaft comprises a 316-stainless steel.

6. The apparatus of claim 1, wherein the malleable portion comprises a shape memory nitinol.

7. The apparatus of claim 1, wherein the malleable portion comprises an annealed stainless steel.

8. The apparatus of claim 1, wherein the malleable portion comprises a flexible polymer supported by a shape memory nitinol wire.

9. The apparatus of claim 1, further comprising a shaping tool configured to bend the malleable portion from the first angular configuration to the second angular configuration.

10. The apparatus of claim 1, wherein the proximal body and the hollow shaft are made from steel.

11. The apparatus of claim 1, wherein a portion of the hollow shaft is dimensioned to be inserted into the nasal cavity.

12. The apparatus of claim 1, wherein the shaft defines a first inner diameter, wherein the malleable portion defines a second inner diameter, wherein the second inner diameter is larger than the first inner diameter.

13. The apparatus of claim 1, wherein the second angular configuration is around 110 degrees.

14. The apparatus of claim 1, wherein the second angular configuration is around 70 degrees.

15. The apparatus of claim 1, wherein the hollow shaft also comprises a malleable material.

16. An apparatus, the apparatus comprising:
(a) a dilation catheter, wherein the dilation catheter comprises an expandable dilator, wherein the expandable dilator is configured to transition between a non-expanded state and an expanded state, wherein the expandable dilator is configured to fit in an anatomical passageway associated with drainage of a paranasal sinus when the expandable dilator is in the non-expanded state, wherein the expandable dilator is configured to dilate an anatomical passageway associated with drainage of a paranasal sinus when the expandable dilator is in the expanded state; and
(b) a guide catheter, wherein the guide catheter comprises:
(i) a hollow shaft, wherein the hollow shaft comprises a first inner diameter having a first dimension,
(ii) an open distal tip, and
(iii) a bent distal portion extending between the hollow shaft and the open distal tip, wherein the bent distal portion comprises a second inner diameter having a second dimension, wherein the first dimension of the first inner diameter is smaller than the second dimension of the second inner diameter,
wherein the hollow shaft, the open distal tip, and the bent distal portion together define a lumen, wherein the lumen includes the first and second inner diameters, wherein the dilation catheter is slidably disposed in the lumen.

17. The apparatus of claim 16, further comprising a tapered transitioning section coupling the bent distal portion with the hollow shaft.

18. The apparatus of claim 16, further comprising a tapered transitioning section coupling the open distal tip with the bent distal portion.

19. The apparatus of claim 16, wherein the bent distal portion has an inner diameter of around 0.110 inches.

20. An apparatus, the apparatus comprising:
(a) a dilation catheter, wherein the dilation catheter comprises an expandable dilator, wherein the expandable dilator is configured to transition between a non-expanded state and an expanded state, wherein the expandable dilator is configured to fit in an anatomical passageway associated with drainage of a paranasal sinus when the expandable dilator is in the non-expanded state, wherein the expandable dilator is configured to dilate an anatomical passageway associated with drainage of a paranasal sinus when the expandable dilator is in the expanded state; and
(b) a guide catheter, wherein the guide catheter comprises:
(i) a proximal body,
(ii) a hollow shaft extending distally from the proximal body, and
(iii) an open distal tip, wherein the open distal tip has a bulbous configuration,
wherein the proximal body, hollow shaft, and distal tip together define a lumen, wherein the dilation catheter is slidably disposed in the lumen,
wherein the proximal body, the hollow shaft, and the open distal tip are made from a metallic material.
